(19) United States
(12) Patent Application Publication
(10) Pub. No.: US 2017/0332985 A1
(43) Pub. Date: Nov. 23, 2017

(54) CEPHALOMETRIC PATIENT POSITIONING UNIT EXTRA ORAL DENTAL IMAGING DEVICES

(71) Applicant: TROPHY, Rochester, NY (US)

(72) Inventors: Philippe Congy, Marne la Vallee Cedex 2 (FR); Olivier Martino, Marne la Vallee Cedex 2 (FR)

(21) Appl. No.: 15/525,601

(22) PCT Filed: Feb. 12, 2015
(86) PCT No.: PCT/IB15/00328

§ 371 Date: May 10, 2017

Related U.S. Application Data
(60) Provisional application No. 62/087,386, filed on Dec. 4, 2014.

(51) Int. Cl. A61B 6/14; A61B 6/06; A61B 6/04

(52) U.S. Cl. CPC A61B 6/14; A61B 6/0492; A61B 6/06

(57) ABSTRACT

A patient positioning unit for an extra-oral imaging system can include a pair of elongated arm extending parallel to a first direction, each elongated arm including an engaging surface along a medial side between a first end and a second end, first and second temporal holding members, each operatively coupled to a second end of one elongated arm, and a sliding member having an engaged surface along at least two contacting sides, the sliding member mounted between the engaging surfaces near the first end of each elongated arm, where each of the engaged surfaces contacts one of the engaging surfaces.

### FIELD OF THE INVENTION

The invention relates generally to the field of dental x-ray imaging, and more particularly, to imaging in a cephalometric x-ray mode for dental applications. Further, the invention relates to a combined cephalometric, panoramic and computed tomography dental imaging apparatus and/or methods.

### BACKGROUND

In the dental imaging field, a cephalometric imaging device includes an x-ray source that emits a conical or pyramidal shaped x-ray beam towards a cephalometric imaging sensor mounted at the end of a long arm. A patient's positioning unit to position the patient's head is located between the x-ray source and the cephalometric sensor at the vicinity of the cephalometric sensor. Then, the x-ray beam originating from the remote x-ray source radiates the patient's skull before impinging the cephalometric sensor. The digitalization of the signal and the treatment of the data lead to the reconstruction of a projection image of the skull or at least a part of the skull of the patient.

In a general dental cephalometric imaging device, a distance between the x-ray source and the cephalometric sensor is typically 1.7 m. As the patient's positioning unit located at the vicinity of the cephalometric sensor and away from the x-ray source, the magnitude ratio of the image size to the patient's skull size is close to 1 for any part of the skull.

The head of the patient has to be very precisely positioned and fixed relative to the sensor for dental cephalometric imaging. Especially for cephalometric profile imaging, the sagittal plane of the patient, which is the plane of symmetry of the patients' head, must be strictly parallel to the plane of the cephalometric sensor, so that the anatomical structures of both halves of the skull precisely superimpose. If the sagittal plane is not parallel to the plane of the cephalometric sensor, the image is of bad quality, can present some structures that do not overlap properly and/or complicate the diagnosis.

Cephalometric patient positioning units according to the prior art usually include two temporal holders terminating with ear rods to be inserted in the ear canals of the patient. In order to allow the patient to insert his head between the two temporal holders, the temporal holders must be separated from each other. Once the head is introduced between both temporal holders, the temporal holders are approached again towards each other so that the ear rods penetrate the ear canals of the patient. According to the prior art, a cumbersome, expensive, and bulky mechanism allows the displacement of both temporal holders at the same time by manipulating only one of the temporal holders. In other words, when the dentist displaces a first temporal holder in one direction, the second temporal holder is automatically displaced in the opposite direction so that the distance between both temporal holders either increases or decreases.

However, there is still a need for a cephalometric patient positioning device that is easy to make, light, simple to use and/or cheap to manufacture.

### SUMMARY

An aspect of this application is to advance the art of medical digital radiography, particularly for dental applications.

Another aspect of this application is to address, in whole or in part, at least the foregoing and other deficiencies in the related art.

It is another aspect of this application to provide, in whole or in part, at least the advantages described herein.

An advantage offered by apparatus and/or method embodiments of the application relates to providing patient positioning device for a dental cephalometric imaging apparatus.

An advantage offered by apparatus and/or method embodiments of the application relates to a light, inexpensive and/or small mechanism with capability to automatically displace a second temporal holder in the opposite direction when the dentist displaces a first temporal holder in one direction so that the distance between the two temporal holders either increases or decreases.

An advantage offered by apparatus and/or method embodiments of the application relates to improved imaging of teeth, jaw and head features or surfaces at a lower cost over conventional imaging methods.

According to one aspect of the disclosure, there is provided an extra-oral system that can include a support base adjustable in at least one dimension; a cephalometric module coupled to the support base and configured to position a cephalometric imaging sensor about a first imaging area formed with an x-ray source, where x-rays from the x-ray source impinge the cephalometric sensor after radiating the first imaging area; the cephalometric module including a collimator; and a cephalometric patient positioning unit positioned operatively near the first imaging area; the patient positioning unit including a pair of elongated arm extending parallel to a first direction, each elongated arm including an engaging surface along a medial side between a first end and a second end, first and second temporal holding members each operatively coupled to a second end of one elongated arm, and a sliding member having an engaged surface along at least two opposing sides, the sliding member mounted between the engaging surfaces near the first end of each elongated arm, where each of the engaged surfaces contacts one of the engaging surfaces.

These objects are given only by way of illustrative example, and such objects may be exemplary of one or more embodiments of the invention. Other desirable objectives and advantages inherently achieved by the may occur or become apparent to those skilled in the art. The invention is defined by the appended claims.

### BRIEF DESCRIPTION OF THE DRAWINGS

The foregoing and other objects, features, and advantages of the invention will be apparent from the following more particular description of the embodiments of the invention, as illustrated in the accompanying drawings.

The elements of the drawings are not necessarily to scale relative to each other. Some exaggeration may be necessary in order to emphasize basic structural relationships or principles of operation. Some conventional components that would be needed for implementation of the described embodiments, such as support components used for providing power, for packaging, and for mounting and protecting system optics, for example, are not shown in the drawings in order to simplify description.

FIG. 1 is a diagram that shows a perspective view of a extra-oral dental imaging system with a cephalometric imaging unit including an exemplary patient positioning unit according to the application.

FIG. 2 is a diagram that shows a perspective view of a cephalometric imaging unit of FIG. 1.

FIG. 3a is a diagram that shows a perspective view of an exemplary patient positioning unit according to the application in a position of the temporal holders corresponding to the smallest gap therebetween.

FIG. 3b is a diagram that shows a perspective view of an exemplary patient positioning unit according to the application in a position of the temporal holders corresponding to the largest gap therebetween.

FIG. 4 is a diagram that shows a view of an exemplary drive mechanism embodiment for a patient positioning unit according to the application.

### DESCRIPTION OF EXEMPLARY EMBODIMENTS

The following is a description of exemplary embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

As used herein, the term “energizable” relates to a device or set of components that perform an indicated function upon receiving power and, optionally, upon receiving an enabling signal. The term “actuable” has its conventional meaning, relating to a device or component that is capable of effecting an action in response to a stimulus, such as in response to an electrical signal, for example.

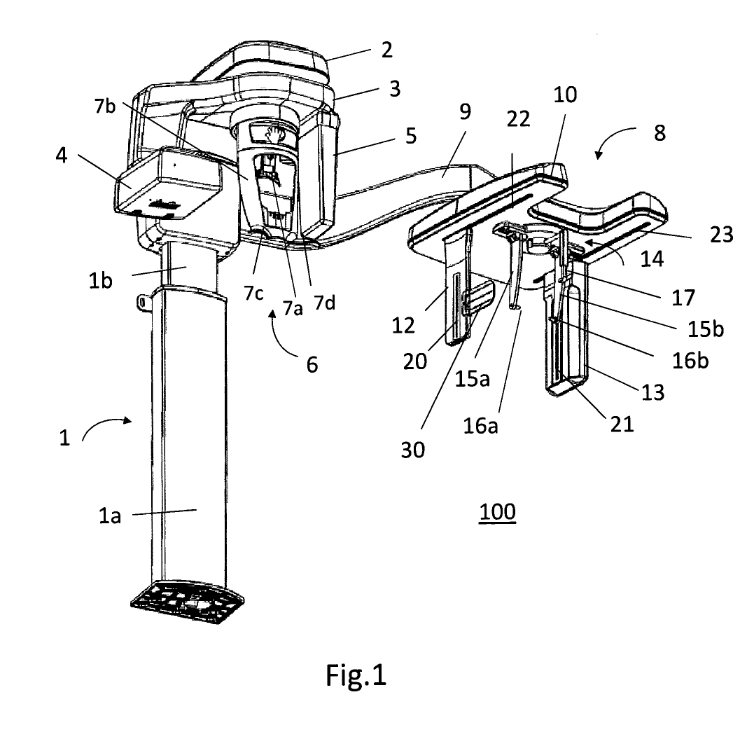

FIG. 1 is a diagram that shows a perspective view of a extra-oral dental imaging system with a cephalometric imaging unit including an exemplary patient positioning unit according to the application. As shown in FIG. 1, an exemplary extra-oral dental imaging system 100 includes a support structure that can include a support column 1. The column 1 may be adjustable in two-dimensions or three-dimensions. For example, the column 1 can be telescopic and may include an upper part 1b sliding inside a lower part 1a. A horizontal mount 2 may be supported or held by the vertical column 1 and can support a rotatable gantry 3. An x-ray source 4 and a first x-ray imaging sensor 5 are attached or coupled to the gantry 3 in correspondence (e.g., opposite, aligned) to each other. The first x-ray sensor 5 may be a panoramic (e.g., slit-shaped) sensor or a Computerized Tomography (e.g., rectangular, square-shaped) sensor. Preferably, the x-ray beam originating from the x-ray source 4 impinges the sensor 5 after radiating a first imaging area or the patient. A first patient positioning and holding system 6 can be operatively positioned near or in the first imaging area. For example, the first patient positioning and holding system 6 may be between the x-ray source 4 and the first x-ray imaging sensor 5. The first patient positioning and holding system 6 can include a forehead support 7a and a shield 7b including two handles 7c and 7d. The patient can then grasp the handles 7c and 7d and remain motionless during the CT scan or panoramic scan.

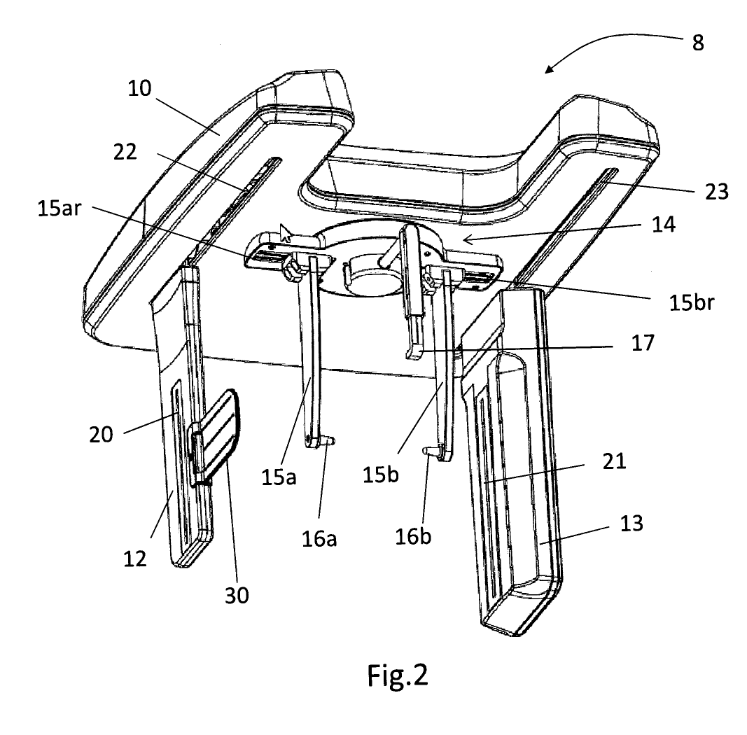

More remote from the x-ray source 4, a cephalometric imaging unit 8 may be held in correspondence to the x-ray source 4. For example, as shown in FIG. 1, the cephalometric imaging unit 8 can be attached or coupled to the upper part 1b of the vertical column via an extended (e.g., horizontal) cephalometric arm 9. The cephalometric imaging unit 8 can include a mount 10 supporting a collimator 12, a second or cephalometric sensor 13 and a second patient positioning and holding system 14. As can also be seen in FIG. 2, the second patient positioning and holding system 14 can include a forehead support 17 and two temporal holding members 15a and 15b each supporting an ear rod 16a and 16b. Preferably, the x-ray beam originating from the x-ray source 4 impinges the sensor 13 after radiating a second or cephalometric imaging area or the patient. The second patient positioning and holding system 14 can be operatively positioned near or in the second imaging area. For example, thanks to the second patient positioning and holding system 14, the patient is precisely and repeatedly positioned between the collimator 12 and the sensor 13, preferably in the second imaging area. One or more of the holding members 15a and 15b can respectively slide along rails 15ar and 15br so that the distance between the two ear rods 16a and 16b can be changed to fit the patient's head. The forehead support 17 can also be adjustable. For example, the forehead support 17 can be adjustable in at least two orthogonal dimensions by sliding along the horizontal and vertical directions. Alternatively, the forehead support 17 can be adjustable in three-dimensions or around three or more orthogonal axis.

As shown in FIG. 2, the collimator 12 can include an elongated opening or slit 20 to shape an x-ray beam. The x-ray sensor 13 can include an active area 21 having an elongated shape (e.g., a vertical slit) facing the vertical slit 20 of the collimator 12 (e.g., across the second imaging area). The sensor 13 and the collimator 12 face each other so that the sensor 13 can receive the x-rays originating from the x-ray source 4 after the x-ray beam was shaped by the collimator 12 and after the x-ray beam radiated the patient positioned and held on the second patient's positioning and holding system 14. For the scanning of the complete skull of the patient, the collimator 12 can move or slide during the x-ray scan along a rail 22 and the sensor 13 can move or slide along the rail 23. Both rails 22, 23 can be coupled to the mount 10. For example, the rails 22, 23 can be embodied on a lower face of the mount 10 of the cephalometric imaging unit 8. At any time during the cephalometric x-ray scan, an alignment may exist between a primary collimator in front of the source (not shown), the slit 20 of the collimator 12 and the active area 21 of the sensor 13. Such x-ray alignment is disclosed, for example, in U.S. Pat. No. 5,511,106. At selected positions of the ensemble during the cephalometric scan, an x-ray digital image is obtained by the sensor 13. At the end of the cephalometric scan, an image reconstructing device (e.g., hardware, software and/or image processing) reconstructs the whole skull image on the basis of the plurality of images obtained during the cephalometric scan, for example using algorithms known to the person skilled in the art.

In the cephalometric or skull imaging technique, the patient can be positioned facing the x-ray beam or in a profile position. As shown in FIGS. 1-2, the second patient positioning and holding system 14 can be rotated 90 degrees for profile position cephalometric imaging. Again, thanks to the second patient positioning and holding system 14, the patient can be precisely and repeatedly positioned between the collimator 12 and the sensor 13, preferably in the second imaging area for profile position cephalometric imaging.

For a correct patient's positioning allowing a good image quality in the exemplary extra-oral dental imaging system 100, the Frankfort plane containing a straight line passing though the bottom of the eye socket and the ear canal must be horizontal. For the purpose of controlling that the Frankfort plane is horizontal, an at least partially transparent visual indicator 30 can be used.

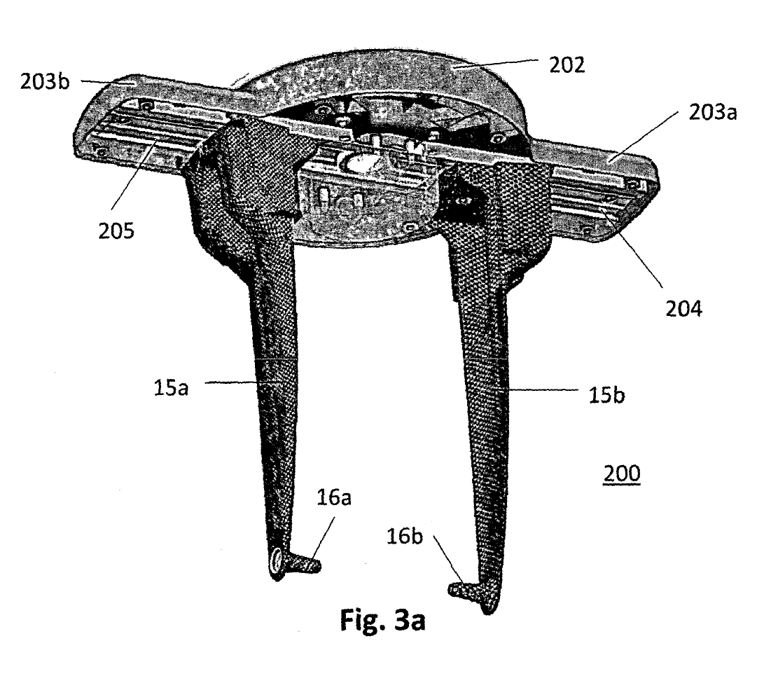

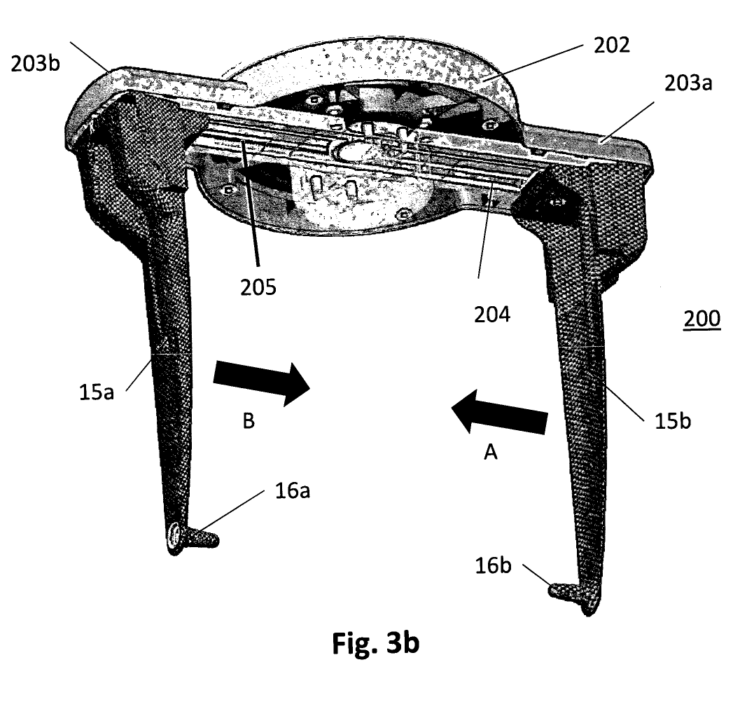

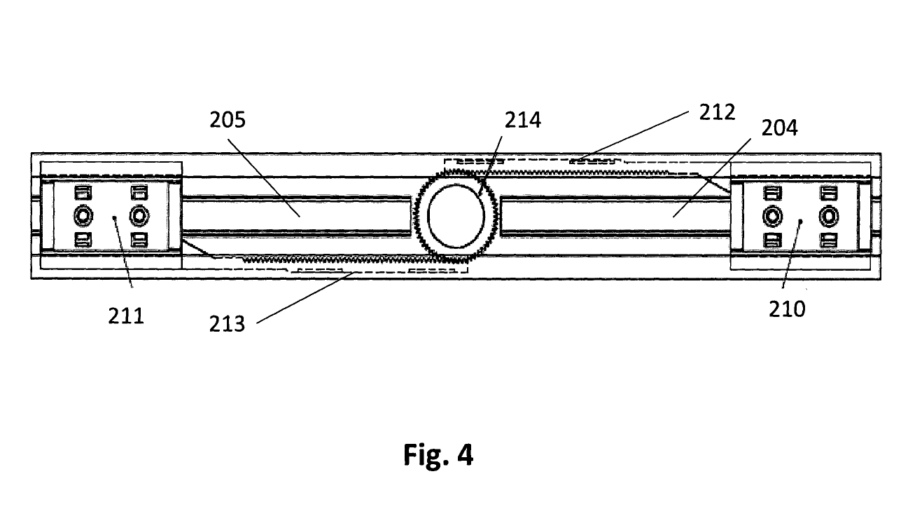

FIGS. 3a-3b are diagrams that shows a perspective view of an exemplary transport mechanism embodiment for a patient positioning unit as shown in FIGS. 1-2 according to the application. As shown in FIGS. 3a-3b, a cephalometric patient positioning unit 200 according to the application can include a mounting unit 202 to fix the positioning unit 200 to a cephalometric mount (e.g., mount 10) (not shown) and, slightly vertically offset from the mounting unit 202, two wings 203a and 203b. Two linear rails 204 and 205 can be incorporated inside the wings 203a and 203b. Two temporal holder supports 210 and 211 (see FIG. 4) can slide reciprocally along the rails 204 and 205 and can hold or support temporal holders 15b and 15a, respectively. Rails 204, 205 can be used for rails 15ar and 15br, respectively. At least portions of a nasion support is represented in transparency in FIG. 3a.

FIG. 4 is a diagram that shows a view of an exemplary drive mechanism embodiment for a patient positioning unit according to the application. Embodiments of the exemplary drive mechanism can provide movement through mechanical interaction (or electro-mechanical interaction) of a second temporal holder in an opposite direction when a first temporal holding member is moved in a selected direction. As shown in FIG. 4, two toothed racks 212 and 213 can be respectively linked to the two temporal holder supports 210 and 211 and cooperate with a toothed wheel 214. The contacts of both toothed racks 212 and 213 with the toothed wheel 214 are preferably diametrically opposed with respect with the toothed wheel 214. In one embodiment, an interlocking portion for the toothed racks 212 and 213 can be on a medial side of the toothed racks 212 and 213. As shown in FIG. 4, engaging surfaces of the toothed racks 212 and 213 and engaged surfaces of the toothed wheel 214 each can include a plurality of protrusions and recesses that interlock when moved. These plurality of protrusions and recesses can have any prescribed shape that generates relative motion therebetween when the two temporal holder supports 210 and 211 are moved. In one embodiment, the two temporal holder supports 210 and 211 can be locked in at least one selected position.

As shown in FIGS. 3a-4, when the dentist grasps a first temporal holder 15b and displaces the first temporal holder 15b in the direction of the arrow A of the FIG. 3b, the temporal holder support 210 slides along the rail 204 in the same direction. The toothed rack 212 is displaced by the same amount of displacement and thanks to the cooperation of the toothed rack 212 with the toothed wheel 214, movement of the toothed rack 212 makes the toothed wheel 214 rotate. Since the second toothed rack 213 also cooperates with the toothed wheel 214, the second toothed rack 213 is displaced in the opposite direction (of the first toothed rack 212), but with the same distance or amount of displacement. The second temporal holder support 211 incurs the same movement of the second toothed rack 213 and consequently the second temporal holder 15a is directed along the direction of the arrow B (see FIG. 3b) towards the first temporal support 15b. The distance between both temporal holders 15b, 15a is then narrowed. FIG. 3a is a diagram that shows a perspective view of an exemplary patient positioning unit according to the application in a position of the temporal holders 15b, 15a corresponding to the smallest gap therebetween. According to certain exemplary embodiments of the application, a distance between both temporal holders can then be changed easily by handling one single temporal holder and using a light, inexpensive and/or small linear rack mechanism. Further, in certain exemplary embodiments, the toothed racks 212, 213 can be mounted in an enclosure to restrict movement of the toothed racks 212, 213 to motions parallel to a first direction or the rails 204, 205. In certain exemplary embodiments, the toothed wheel 214 can be contained in an enclosure to restrict movement to a translational or rotational movement between the toothed racks 212, 213.

Consistent with exemplary embodiments of the application, a computer program utilizes stored instructions that perform on image data that is accessed from an electronic memory. As can be appreciated by those skilled in the image processing arts, a computer program for operating the imaging system in an exemplary embodiment of the present application can be utilized by a suitable, general-purpose computer system, such as a personal computer or workstation. However, many other types of computer systems can be used to execute the computer program of the present application, including an arrangement of networked processors, for example. The computer program for performing exemplary methods/apparatus of the present application may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. The computer program for performing exemplary methods/apparatus of the present application may also be stored on computer readable storage medium that is connected to the image processor by way of the internet or other network or communication medium. Those skilled in the art will further readily recognize that the equivalent of such a computer program product may also be constructed in hardware.

It should be noted that the term “memory”, equivalent to “computer-accessible memory” in the context of the present disclosure, can refer to any type of temporary or more enduring data storage workspace used for storing and operating upon image data and accessible to a computer system, including a database, for example. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer is also considered to be a type of memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It will be understood that the computer program product of the present application may make use of various image manipulation algorithms and processes that are well known. It will be further understood that the computer program product embodiment of the present application may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the present application, are not specifically shown or described herein and may be selected from such algorithms, systems, hardware, components and elements known in the art.

In certain exemplary embodiments, the first patient positioning and holding system 6 can include a substantially transparent shield suspended from the mount 2 or the rotatable gantry 3, and a chin positioning element that can include a chin rest and a bite element. In one embodiment, the shield can include an open window disposed between a chin support and a forehead support. In one embodiment, the shield can be visibly transparent, transparent to additional radiation including x-rays and/or formed from a molded polycarbonate material. In one embodiment, the chin support can include a height adjuster for the bite element and the forehead support is configured to be adjustably pivotable toward the patient. In one embodiment, the shield can include one or more controls for setting a column height adjustment for the mount on the shield or mounted on a separate panel that is coupled to the shield. In one embodiment, the shield can include one or more markings to assist in patient positioning. In one embodiment, the shield can include a first Frankfort plane positioning indicator.

In certain exemplary embodiments, an extra-oral imaging system can include a support base adjustable in at least one dimension; a first mount mounted to the support base and configured to revolve an x-ray source and an imaging sensor panel about an imaging area; and a first patient positioning unit coupled to the extra-oral imaging system and positioned between the x-ray source and first sensor so that x-rays impinge the first sensor after radiating the imaging area, including a chin support coupled to the first patient positioning unit and includes a chin positioning element; a head support coupled to the first patient positioning unit shield; and a first Frankfort plan positioning indicator; a second mount mounted to the support base and configured to position a second imaging sensor panel about a second imaging area; and a second patient positioning unit coupled to the second mount and positioned between the x-ray source and the second sensor so that x-rays impinge the second sensor after radiating a second imaging area including a head support coupled to the second patient positioning unit; and a second Frankfort plane positioning indicator. In one embodiment, the second Frankfort plane positioning indicator is fixedly mounted, detachably mounted, or mounted to move between at least two positions, or rotatably mounted. In one embodiment, the second patient positioning unit is configured to repeatably and accurately position a patient between the x-ray source and the second imaging sensor panel.

The invention has been described in detail, and may have been described with particular reference to an exemplary or presently preferred embodiment, but it will be understood that variations and modifications can be effected within the spirit and scope of the invention. For example, embodiments of a patient positioning unit have been described having an amount of movement of toothed racks being equal to an amount of movement by temporal holding members. Alternatively, the toothed racks and/or sliding member can be configured to move a different amount (e.g., greater amount) than the amount of movement by the temporal holding members. Further, embodiments of a patient positioning unit have been described with a circular toothed wheel 214, however, embodiments are not intended to be so limited as alternative shapes (e.g., elliptical, octagon, linear, non-linear, etc.) can be used for the element 214. In one embodiment, an entire rail assembly can be rectangular with a height of less than 3 cm or preferably less than 1 cm. In addition, exemplary apparatus and/or method embodiments according to the application have been described relative to a combined cephalometric, panoramic and computed tomography dental imaging apparatus, but are intended to be applicable to stand-alone cephalometric imaging apparatus or cephalometric imaging apparatus with any additional mode(s) of operation or functionality. The presently disclosed exemplary embodiments are therefore considered in all respects to be illustrative and not restrictive. The scope of the invention is indicated by the appended claims, and all changes that come within the meaning and range of equivalents thereof are intended to be embraced therein.

While the invention has been illustrated with respect to one or more implementations, alterations and/or modifications can be made to the illustrated examples without departing from the spirit and scope of the appended claims. In addition, while a particular feature of the invention can have been disclosed with respect to one of several implementations, such feature can be combined with one or more other features of the other implementations as can be desired and advantageous for any given or particular function. The term “at least one of” is used to mean one or more of the listed items can be selected. The term “about” indicates that the value listed can be somewhat altered, as long as the alteration does not result in nonconformance of the process or structure to the illustrated embodiment. Finally, “exemplary” indicates the description is used as an example, rather than implying that it is an ideal. Other embodiments of the invention will be apparent to those skilled in the art from consideration of the specification and practice of the invention disclosed herein. It is intended that the specification and examples be considered as exemplary only, with a true scope and spirit of the invention being indicated by the following claims, and all changes that come within the meaning and range of equivalents thereof are intended to be embraced therein.

1. An extra-oral imaging system, the extra-oral system comprising:
a support base adjustable in at least one dimension;
a cephalometric module coupled to the support base and configured to position a cephalometric imaging sensor about a first imaging area formed with an x-ray source, where x-rays from the x-ray source impinge the cephalometric sensor after radiating the first imaging area; the cephalometric module comprising a collimator; and
a cephalometric patient positioning unit positioned operatively near the first imaging area; the patient positioning unit comprising
a pair of elongated arm extending parallel to a first direction, each elongated arm including an engaging surface along a medial side between a first end and a second end,
first and second temporal holding members, each operatively coupled to a second end of one elongated arm, and
a sliding member having an engaged surface along at least two opposing sides, the sliding member mounted between the engaging surfaces near the first end of each elongated arm, where each of the engaged surfaces contacts one of the engaging surfaces.

2. The extra-oral imaging system of claim 1, where movement of the first temporal holder in the first direction through mechanical interaction causes the second temporal holding member to move in a direction opposite the first direction.

3. The extra-oral imaging system of claim 2, where the engaging surfaces and the engaged surfaces each comprises a plurality of protrusions and recesses that interlock when an elongated arm moves in the first direction or moves in a second direction opposite to the first direction.

4. The extra-oral imaging system of claim 1, where the elongated arms are mounted in an enclosure to restrict movement of the elongated arm to motions parallel to the first direction.

5. The extra-oral imaging system of claim 1, where the sliding member is contained in an enclosure to restrict movement to rotational movement between the pair of elongated arms.

6. The extra-oral imaging system of claim 1, where each elongated arm is connected by a mounting structure configured to slide along a rail to one of the temporal holding members.

7. The extra-oral imaging system of claim 1, where the patient positioning unit is mounted between the collimator and the cephalometric imaging sensor.

8. The extra-oral imaging system of claim 1, where the first direction is orthogonal to an imaging plane of the cephalometric imaging sensor.

9. The extra-oral imaging system of claim 1, where the cephalometric patient positioning unit comprises:
a forehead support, the forehead support is adjustable in at least two dimensions; and
the two temporal holding members adjustable in at least one dimension; and
a retractable cephalometric Frankfort plane positioning indicator, where the retractable cephalometric Frankfort plane positioning indicator is configured to be mounted to one or both sides of the integral cephalometric collimator.

10. The extra-oral imaging system of claim 1, comprising:
a first mount mounted to the support base and configured to revolve the x-ray source and an imaging sensor about a second imaging area so that x-rays impinge the imaging sensor after radiating the second imaging area; and
a second patient positioning unit coupled to the extra-oral imaging system and positioned operatively near the second imaging area, comprising:
an elongated shield comprising handles;
a chin support coupled to the elongated shield and comprising a chin positioning element;
a head support coupled to the elongated shield; and
a second Frankfort plan positioning indicator.
